(19) United States
(12) Patent Application Publication
(10) Pub. No.: US 2007/0218149 A1
(43) Pub. Date: Sep. 20, 2007

(54) Composition for treating joint or muscle pain

(75) Inventor: Richard H. Blau, Locust Valley, NY (US)

Correspondence Address: HOFFMANN & BARON, LLP, 6900 JERICHO TURNPIKE, SYOSSET, NY, 11791, US

(73) Assignee: Medicomp Systems, Inc.

(21) Appl. No.: 11/717,979

(22) Filed: Mar. 14, 2007

Related U.S. Application Data
(60) Provisional application No. 60/782,510, filed on Mar. 15, 2006.

(51) Int. Cl. A61K 36/906; A01N 37/36

(52) U.S. Cl. 424/756; 424/773; 514/163

(57) ABSTRACT

The present invention relates to treatment of joint or muscle pain and, in particular, combinations of ingredients for such treatment. The invention provides a composition, which includes the following components: emu oil, a salicylate analgesic agent, devil's claw, and ginger. The components are present in the composition in an amount sufficient to alleviate joint or muscle pain in a mammal in need thereof. A composition having essentially emu oil, a salicylate analgesic agent, devil's claw and ginger is also provided. The invention further provides a method for alleviating joint or muscle pain in a mammal. The method includes administering topically to the mammal the composition of the present invention.

The application asserts priority to U.S. Provisional Application Ser. No. 60/782,510 filed on Mar. 15, 2006. The specification of U.S. Provisional Application Ser. No. 60/782,510 is hereby incorporated by reference.

### BACKGROUND OF THE INVENTION

The invention relates to treatment of joint or muscle pain and, in particular, combinations of ingredients for such treatment.

Many people experience joint pain and/or muscle pain in various areas of the body, such as the back, neck, hand and foot extremities, the knees, elbows and shoulders, etc. The pain causes considerable discomfort, and costs billions of dollars in lost days of work. Symptoms of joint or muscle pain include inflammation, lameness and loss of mobility.

### SUMMARY OF THE INVENTION

The invention provides a composition which, includes the following components: (i) emu oil; (ii) a salicylate analgesic agent; (iii) devil's claw; and (iv) ginger. The components are present in the composition in an amount sufficient to alleviate joint pain or muscle pain in a mammal in need thereof.

The composition can contain at least about 2.5%, about 5%, about 7%, or about 12% emu oil by weight. In one embodiment, the composition contains about 13% emu oil by weight. The maximum amount of emu oil present in the composition is about 50%, about 35%, about 30%, about 25%, or about 20% by weight.

The salicylate analgesic agent can be present in the composition in an amount of at least about 2.5%, about 5%, about 7%, or about 12% by weight. In one embodiment, the composition contains about 13% salicylate analgesic agent by weight. The maximum amount of salicylate analgesic agent present in the composition is about 50%, about 35%, about 30%, about 25%, or about 20% by weight.

In one embodiment the salicylate analgesic agent is trolamine. In another embodiment, the salicylate analgesic agent is acetylsalicylic acid.

Devil's claw can be present in the composition in an amount of at least about 0.1%, about 0.5%, or 0.75% by weight. In one embodiment, the composition contains about 1% devil's claw by weight. The maximum amount of devil's claw present in the composition can be about 5%, about 4%, about 3%, or about 2% by weight.

Ginger can be present in the composition in an amount of at least about 0.1%, about 0.5%, or 0.75% by weight. In one embodiment, the composition contains about 1% ginger by weight. The maximum amount of ginger present in the composition can be about 5%, about 4%, about 3%, or about 2% by weight.

In another aspect of the invention, a composition is provided having essentially emu oil, a salicylate analgesic agent, devil's claw and ginger. In this embodiment, other ingredients may be included, so long as they do not affect the characteristics and effect of the four essential ingredients set forth above.

In yet another aspect of the invention, a method is provided for alleviating joint pain or muscle pain in a mammal. The method includes administering topically to the mammal in need thereof the composition of the present invention.

As a result of the present invention, a composition and method are provided containing natural active components for treating joint pain and muscle pain having superior analgesic and anti-inflammatory properties.

For a better understanding of the present invention, together with other and further advantages, reference is made to the following detailed description, and its scope will be pointed out in the claims.

### DETAILED DESCRIPTION OF THE INVENTION

In one aspect of the invention, a composition is provided containing the following components: (i) emu oil; (ii) a salicylate analgesic agent; (iii) devil's claw; and (iv) ginger. The components are present in the composition in an amount sufficient to alleviate joint pain or muscle pain in a mammal in need thereof.

Emu oil is obtained from the fat of an emu, which is a large, flightless Australian bird (Dromiceius novaehollandiae) related to, and resembling, an ostrich. The fat is removed from the emu, and processed to obtain emu oil.

Emu fat can be processed by any method known to those skilled in the art. For example, a cold rendering process can be employed. An example of a cold rendering process for processing emu fat to oil is disclosed in U.S. Pat. No. 6,103,246 to Tisdale et al. The cold rendering process disclosed in U.S. Pat. No. 6,103,246 is hereby incorporated by reference in its entirety.

Emu oil generally contains myristic acid, palmitic acid, palmitoleic acid, margaric acid, stearic acid, elaidic acid, oleic acid, vaccenic acid, linoleic acid, linolenic acid, arachidic acid, and eicosenoic acid.

Preferably, the emu oil is purified. The term “purified” as used herein means that the emu oil contains essentially a biologically active fraction. For example, a biologically active component of emu oil is described in U.S. Pat. No. 5,431,924 to Ghosh, et al.

Preferably, the emu oil is at least about 90% to about 99.9% pure. More preferably, the emu oil is at least about 95% to about 99.9% pure. Optimally, the emu oil is at least about 99% to about 99.9% pure.

Throughout this specification, certain parameters are defined by maximum and minimum amounts. Each minimum amount can be combined with each maximum amount to define a range for the given parameter.

The emu oil component can be present in the composition in an amount of at least about 2.5% by weight, more preferably at least about 5% by weight, even more preferably at least about 7% by weight, and can even be present in the composition in an amount of at least about 12% by weight. For example, the composition can contain about 13% emu oil by weight.

The maximum amount of emu oil component present in the composition can be at most about 50% by weight, more preferably at most about 35% by weight, even more preferably at most about 30% by weight, still more preferably at most about 25% by weight, and yet still more preferably at most about 20% by weight.

The emu oil has both analgesic and anti-inflammatory properties. Also, emu oil can act as a transporter through the skin. Thus, the emu oil in the composition can provide not only its own beneficial effects, but also permit the other components to penetrate the skin, thereby resulting in a greater effect.

Salicylates generally possess analgesic properties. Any salicylate analgesic known to those skilled in the art can be employed. Examples of salicylates include, but are not limited to trolamine (i.e., triethanolamine), acetylsalicylic acid, and combinations thereof. Preferably, the salicylate analgesic agent is trolamine.

The salicylate analgesic agent component can be present in the composition in an amount of at least about 2.5% by weight, more preferably at least about 5% by weight, even more preferably at least about 7% by weight, and can even be present in the composition in an amount of at least about 12% by weight. For example, the composition can contain about 13% salicylate analgesic agent by weight.

The maximum amount of salicylate analgesic agent component present in the composition can be at most about 50% by weight, more preferably at most about 35% by weight, even more preferably at most about 30% by weight, still more preferably at most about 25% by weight, and yet still more preferably at most about 20% by weight.

Devil's claw (i.e., Harpagophytum procumbens) is a native plant of generally southern Africa, especially the Kalahari Desert, Namibia and the island of Madagascar. The name devil's claw is derived from the herb's unusual fruits, which are covered with numerous small claw-like appendages.

Devil's claw is useful in the composition of the present invention, and can be obtained from the whole plant or any component of the plant, such as, for example, the fruit, flowers, leaves, stems, roots, and tuber. Devil's claw contains iridoid glycosides, which are substances believed to be responsible for the herb's anti-inflammatory and analgesic actions. Preferably, the secondary storage roots, or tubers, of the plant, are used in the composition of the present invention.

Any form of devil's claw can be incorporated in the composition of the present invention. Examples of suitable forms of devil's claw include devil's claw powder, devil's claw extracts (e.g., devil's claw powder extracts, devil's claw fluid extracts), one or more active components of devil's claw, parts of or whole devil's claw plants, and mixtures thereof. Preferably, the devil's claw component of the composition is in the form of devil's claw extract or devil's claw powder.

Methods for preparing various forms of devil's claw are well known to those skilled in the art. For example, devil's claw powder can be prepared by cutting the devil's claw, grinding it, and drying the ground devil's claw. A method for extracting the active components of devil's claw is disclosed in U.S. Pat. No. 6,197,307 to Wheatley et al. The extraction method disclosed in U.S. Pat. No. 6,197,307 is hereby incorporated by reference in its entirety.

The devil's claw can be present in the composition in an amount of at least about 0.1% by weight, more preferably at least about 0.5% by weight, even more preferably at least about 0.75% by weight. For example, the composition can contain about 1% devil's claw by weight.

The maximum amount of devil's claw component present in the composition can be at most about 5% by weight, more preferably at most about 4% by weight, even more preferably at most about 3% by weight, and still more preferably at most about 2% by weight.

Ginger usually refers to the underground stem, or rhizome, of the plant Zingiber officinale. The active components of the ginger root are thought to be the volatile oils and pungent phenol compounds (e.g., gingerols and shogaols). These components are believed to contribute to ginger's medicinal properties.

Any form of ginger can be incorporated in the composition of the present invention. Suitable forms include ginger extracts such as ginger powder extracts, ginger fluid extracts, ginger oil, ginger powder, and one or more active components of ginger; parts of or whole ginger plants; and mixtures thereof. Preferably, the ginger component of the composition is in the form of ginger extract or ginger powder.

Methods for preparing various forms of ginger are well known to those skilled in the art. For example, ginger powder can be prepared by cutting the ginger rhizomes, grinding it, and drying the ground ginger. A method for extracting the active components of ginger is disclosed in U.S. Pat. No. 6,596,313 to Rosenbloom. The extraction method disclosed in U.S. Pat. No. 6,596,313 is hereby incorporated by reference in its entirety.

The ginger component can be present in the composition in an amount of at least about 0.1% by weight, more preferably at least about 0.5% by weight, even more preferably at least about 0.75% by weight. For example, the composition can contain about 1% ginger by weight.

The maximum amount of ginger component present in the composition can be at most about 5% by weight, more preferably at most about 4% by weight, even more preferably at most about 3% by weight, and still more preferably at most about 2% by weight.

The emu oil, salicylate analgesic agent, devil's claw and ginger are combined to obtain the composition of the present invention. Any method known to those skilled in the art can be employed to combine the components of the composition. For example, appropriate amounts of each component can be mixed together.

The components of the composition are present in the composition in an amount sufficient to alleviate joint or muscle pain in a mammal in need thereof. Such amounts can be readily determined by physicians and clinicians during, for example, pre-clinical and clinical trials.

The composition of the present invention is suitable for topical administration. Suitable formulations for topical administration include, for example, emollients, creams and lotions.

The composition of the present invention may further include a variety of pharmaceutical or cosmetic ingredients insofar as they do not interfere with the joint pain or muscle pain alleviating effects of the composition.

For example, the composition can optionally contain excipients. Examples of excipients include water, surfactants, emulsifiers, diglycerides, triglycerides, stabilizing agents, antioxidants, glycerol, ethanol, propanol, isopropanol, butanol, polymeric gelling agents, and other formulation components commonly used in the art of pharmaceutical and cosmetic compositions. The composition can also optionally contain emollients, thickeners, preservatives, coloring agents, and fragrance, such as those commonly used in the art of pharmaceutical and cosmetic compositions.

In another aspect, the present invention provides a method for alleviating joint pain or muscle pain in a mammal. The method includes administering topically to the mammal in need thereof, the composition described above.

Joint pain generally refers to stiffness or pain affecting a joint. The stiffness or pain can be caused by many types of injuries or conditions, such as unusual exertion or overuse, including strains or sprains; injury; arthritis, including osteoarthritis, septic arthritis, and rheumatoid arthritis; tendonitis; bursitis; osteomyelitis; chondromalacia patellae; lupus; and infectious diseases, including influenza, measles, rheumatic fever, Epstein-Barr viral syndrome, hepatitis, mumps, rubella, and varicells.

Muscle pain generally refers to aches and pain in a muscle. Muscle pain can also involve ligaments, tendons, and fascia, the soft tissues that connect muscles, bones, and organs together. Examples of causes of muscle pain include, for example, tension or stress; overuse of a muscle (e.g., using a muscle too much, too soon, too often, etc.); injury or trauma, including sprains and strains; polymyositis; dermatomyositis; lupus; fibromyalgia; polymyalgia rheumatica; infections, such as an abscess in a muscle, trichinosis, influenza, Lyme disease, malaria, Rocky mountain spotted fever, polio, etc.; electrolyte imbalances such as too much potassium or calcium; and rhabdomyolsis.

Mammals in need of alleviating joint pain or muscle pain include those mammals suffering from the joint pain or muscle pain described above. Suitable mammals include humans, farm animals, domestic animals, laboratory animals, etc. Some examples of farm animals include cows, pigs, horses, goats, etc. Some examples of domestic animals include dogs, cats, etc. Some examples of laboratory animals include rats, mice, rabbits, guinea pigs, etc.

Joint pain or muscle pain is considered to be alleviated if the pain is reduced by at least about 10%, preferably at least about 25%, more preferably at least about 50%, even more preferably at least about 75%, and even more preferably at least about 90%. Optimally, the composition of the present invention completely alleviates the joint pain or muscle pain.

Very often, alleviation of the pain will be temporary. Therefore, the composition can be reapplied. For example, the composition can be applied one, two, three, four, five, or six or more times a day.

The features and advantages of the compositions described herein are more fully shown by the following examples, which are provided for purposes of illustration, and are not to be considered as limiting the invention in any way.

### EXAMPLES

### Example 1

### A Topical Composition of the Present Invention

A joint pain and muscle pain alleviating topical composition of the present invention containing the following ingredients was prepared:

| | | |
|---|---|---|
| 100 | grams | distilled water |
| 30 | grams | certified and purified Emu oil |
| 27 | grams | lotion base (cetearyl isononanoate, ceteareth-20, |
| | | cetearyl alcohol, glyceryl stearate, glycerin, ceteareth-12, |
| | | cetyl palmitate) |
| 22 | grams | polymer thickener SP (sodium polyacrylate, propylene |
| | | glycol) |
| 5 | grams | germaben II (propylene glycol, diazolidinyl urea, |
| | | methylparaben, propylparaben) |
| 3 | grams | devil's claw (Harpagophytum procumbens) |
| 3 | grams | ginger root |
| 30 | grams | trolamine salicylate |
| 3 | drops | teal (green 5, yellow 5, violet 5) |
| 5 | drops | fragrance |

The composition was prepared by blending, at room temperature, the distilled water, emu oil, and lotion base. The remaining ingredients were added individually with continuous mixing at room temperature.

### Example 2

### Treatment of Localized Pain

Test group: A test group of six subjects with musculoskeletal symptoms were treated with the inventive composition described in Example 1. The six subjects included two human males and four human females. The ages of the patients ranged from 13 to 66 years old. The diagnosis for each is set forth in Table 1 as follows:

TABLE 1
Demographics

| Subject | Musculoskeletal | | | |
|---|---|---|---|---|
| # | Problem | Body Area | Gender | Age |
| 1 | Osteoarthritis | Knee | Male | 52 |
| 2 | Osteoarthritis | Knee | Female | 55 |
| 3 | Muscle Strain | Shoulder | Male | 55 |
| 4 | Meniscal Strain | Knee | Female | 50 |
| 5 | Jaw Pain | Temporomandibular Joint | Female | 51 |
| 6 | Cervical Strain | Neck | Female | 48 |

Pain: All of the subjects had symptoms for at least 24 hours, and were not aware of specific trauma. Prior to administration of the inventive composition, all subjects were asked to use a visual pain scale and grade their pain from 0 to 10 (0 being none; 10 being severe). Their respective grades of pain is set forth in Table 2 as follows:

TABLE 2
Pain Scale Prior to Administration

| Subject # | Pain Scale |
|---|---|
| 1 | 5 |
| 2 | 7 |
| 3 | 4 |
| 4 | 6 |
| 5 | 5 |
| 6 | 6 |

Procedure: All of the subjects were given 1 ml of the inventive composition and asked to massage the composition directly on the skin over the entire painful area for 30 seconds. At the end of five minutes, the subjects were then asked to rank their pain using the visual pain scale described above. The results is set forth in Table 3 as follows:

TABLE 3
Pain Scale Subsequent to Administration

| Subject # | Pain Scale |
|---|---|
| 1 | 0 |
| 2 | 2 |
| 3 | 1 |
| 4 | 1 |
| 5 | 2 |
| 6 | 0 |

Results: All of the subjects found significant relief from minor musculoskeletal pain. The pain relief was observed within 5 minutes of the application of the composition and lasted up to 24 hours. Two subjects (#1 and #6) had complete resolution of their pain. Two subjects (#3 and #4) had pain recurrence 24 hours later. They were given a placebo (base cream without trolamine salicylate, emu oil, ginger extract, and devil's claw) and found no improvement in their pain. They were later given active cream and found improvement in their pain.

The study above demonstrates the effectiveness of the composition of Example 1 in temporary relief of musculoskeletal pain.

What is claimed is:

1. A composition comprising the following components:
a. emu oil;
b. a salicylate analgesic agent,
c. devil's claw; and
d. ginger;
wherein said components are present in an amount sufficient to alleviate joint or muscle pain by topically applying said composition to a mammal in need thereof.

2. A composition according to claim 1, wherein the composition comprises from about 2.5% to about 50% emu oil by weight.

3. A composition according to claim 1, wherein the composition comprises from about 5% to about 35% emu oil by weight.

4. A composition according to claim 1, wherein the composition comprises from about 7% to about 30% emu oil by weight.

5. A composition according to claim 1, wherein the composition comprises from about 12% to about 25% emu oil by weight.

6. A composition according to claim 1, wherein the composition comprises from about 12% to about 20% emu oil by weight.

7. A composition according to claim 1, wherein the composition comprises about 13% emu oil by weight.

8. A composition according to claim 1, wherein the composition comprises from about 2.5% to about 50% salicylate analgesic agent by weight.

9. A composition according to claim 1, wherein the composition comprises from about 5% to about 35% salicylate analgesic agent by weight.

10. A composition according to claim 1, wherein the composition comprises from about 7% to about 30% salicylate analgesic agent by weight.

11. A composition according to claim 1, wherein the composition comprises from about 12% to about 25% salicylate analgesic agent by weight.

12. A composition according to claim 1, wherein the composition comprises from about 12% to about 20% salicylate analgesic agent by weight.

13. A composition according to claim 1, wherein the composition comprises about 13% salicylate analgesic agent by weight.

14. A composition according to claim 1, wherein the salicylate analgesic agent is trolamine salicylate.

15. A composition according to claim 1, wherein the salicylate analgesic agent is acetylsalicylic acid.

16. A composition according to claim 1, wherein the salicylate analgesic agent is methyl salicylate.

17. A composition according to claim 1, wherein the compositions comprises from about 0.1% to about 5% devil's claw by weight.

18. A composition according to claim 1, wherein the compositions comprises from about 0.5% to about 4% devil's claw by weight.

19. A composition according to claim 1, wherein the compositions comprises from about 0.75% to about 3% devil's claw by weight.

20. A composition according to claim 1, wherein the compositions comprises from about 0.75% to about 2% devil's claw by weight.

21. A composition according to claim 1, wherein the composition comprises about 1% devil's claw by weight.

22. A composition according to claim 1, wherein the compositions comprises at least about 0.1% to about 5% ginger by weight.

23. A composition according to claim 1, wherein the compositions comprises at least about 0.5% to about 4% ginger by weight.

24. A composition according to claim 1, wherein the compositions comprises at least about 0.75% to about 3% ginger by weight.

25. A composition according to claim 1, wherein the compositions comprises at least about 0.75% to about 2% ginger by weight.

26. A composition according to claim 1, wherein the composition comprises about 1% ginger by weight.

27. A composition consisting essentially of the following components:
a. emu oil;
b. a salicylate analgesic agent,
c. devil's claw; and
d. ginger;
wherein said components are present in an amount sufficient to alleviate joint or muscle pain by topically applying said composition to a mammal in need thereof.

28. A method for alleviating joint or muscle pain in a mammal in need thereof comprising administering topically to the mammal a composition comprising the following components:
a. emu oil;
b. a salicylate analgesic agent,
c. devil's claw; and
d. ginger;
wherein said components are present in an amount sufficient to alleviate said joint or muscle pain in said mammal.
